(19) United States
(12) Patent Application Publication
(10) Pub. No.: US 2023/0405098 A1
(43) Pub. Date: Dec. 21, 2023

(54) TYPE E BOTULINUM TOXIN TO TREAT BOTULISM

(71) Applicant: InnoMed Technologies, Inc., Encino, CA (US)

(72) Inventor: John Joseph, Westlake Village, CA (US)

(73) Assignee: InnoMed Technologies, Inc., Encino, CA (US)

(21) Appl. No.: 18/313,312

(22) Filed: May 5, 2023

Related U.S. Application Data
(60) Provisional application No. 63/353,017, filed on Jun. 16, 2022.

(51) Int. Cl. A61K 38/48; A61K 9/00; A61K 45/06

(52) U.S. Cl. CPC A61K 38/4893; A61K 9/0019; A61K 45/06

(57) ABSTRACT

Botulism is a potentially fatal illness caused by a toxin produced by the bacterium Clostridium botulinum. There are seven main types of botulinum toxin but the most common is type A (BoNT/A). There are few treatments for botulism which are often limited to supportive efforts. Embodiments include methods of treating botulism using Type E Botulinum toxin (BoNT/E). Intravenous administration allows administration of large doses of BoNT/E to systemically treat the disease and improve recovery times.

### RELATED APPLICATIONS

The present application claims priority to U.S. Provisional Application No. 63/353,017 filed Jun. 16, 2022, the contents of which are incorporated herein by reference.

### FIELD OF THE INVENTION

The invention relates to therapeutics, and more specifically, it relates to the use of Type E Botulinum toxin to treat botulism.

### BACKGROUND

Botulism is a disease of vertebrate animals, including humans, caused by a toxin produced by the bacterium Clostridium botulinum. Botulism can be spread in several ways. The bacterial spores that cause the disease are common in both soil and water. They produce the botulinum toxin when exposed to low oxygen levels and certain temperatures. Foodborne botulism can occur when food containing the toxin is eaten. Infant botulism can occur when the bacteria develops in the intestines and releases the toxin. Wound botulism is found most often among those who inject illicit drugs. In this situation, spores enter a wound, and in the absence of oxygen, release the toxin. The diagnosis of botulism can be confirmed by identifying the toxin or bacteria in the person in question.

Botulism is a potentially fatal illness. Initial symptoms can include weakness, blurred vision, fatigue and difficulty speaking. Weakness of the arms, chest muscles and legs can follow. Vomiting, swelling of the abdomen, and diarrhea may also occur. Supportive care for botulism includes monitoring respiratory function. Respiratory failure due to paralysis may require mechanical ventilation plus intensive medical and nursing care. In some abdominal cases, physicians may try to remove contaminated food that remains in the digestive tract by inducing vomiting or using enemas. Wounds can be treated to remove the source of the toxin-producing bacteria.

The toxin that causes botulism is Botulinum toxin (“BoNT”) and is commonly referred to as “Botox.” It is a neurotoxic protein that prevents the release of the neurotransmitter acetylcholine from axon endings at the neuromuscular junction, thus causing flaccid paralysis. The toxin is used commercially for therapeutic and cosmetic purposes. Injections with botulinum toxin are effective for many clinical disorders that involve involuntary muscle activity or increased muscle tone. It is also the most common cosmetic procedure performed world-wide, with estimates of nearly three million injections per year.

Depending on the target tissue, BoNT can block the cholinergic autonomic innervation of the tear, salivary, and sweat glands or the cholinergic neuromuscular innervation of striated and smooth muscles. After intramuscular injection, the dose-dependent paralytic effect of BoNT can be detected within two to three days. It reaches its maximal effect in less than two weeks and gradually begins to decline in a few months due to the ongoing turnover of the synapses at the neuromuscular junction. The duration of effect lasts somewhere between three months and six months, and the benefits have been observed to increase with time. There has been no evidence of any long-term or permanent degeneration or atrophy of muscles in patients with repeated injections of BoNT over an extended period. Intoxication of the nerve terminal by BoNT is fully reversible and does not lead to neurodegeneration. Upon synaptic blockade of cholinergic nerve terminals by therapeutic BoNT, the neuron forms new synapses that replace its original ones in a process known as sprouting. As the nerve terminals eventually recover, original synapses are regenerated, the sprouts retreat, and the synaptic contact is reestablished leading to restoration of exocytosis.

Although BoNT is often used therapeutically, its use presents risks. Injections of botulinum toxin may be responsible for severe side effects. Patients are subjected to the possibility of systemic and generalized botulinum toxin diffusion. And although food safety measures and improved hygiene have reduced the incidence of botulism, the disease remains a threat. Further, botulism infections are relatively common in cattle and other livestock. Botulinum toxins also pose a threat as they may be used as bioterrorism agents or in biological warfare.

There are few treatments for botulism. Treatments are often limited to supportive efforts. These supportive efforts typically include administering an antibiotic and monitoring symptoms. Antitoxins have been proposed but have limitations. The development of monoclonal antibody (mAb) therapy for botulism is complicated by the fact that there are at least seven BoNT serotypes (A-G) that show little, if any, antibody cross-reactivity. Vaccines have been proposed but also have shortcomings.

Accordingly, there is a need for improved methods to treat botulism. The methods should be safe and effective with minimal side effects. Aspects of the present disclosure fulfill these needs and provide further related advantages as described in the following summary.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Embodiments include pharmaceutical formulation containing Type E Botulinum toxin (BoNT/E) for therapeutic use.

Embodiments include a composition of Type E Botulinum toxin (BoNT/E) for use in the treatment of botulism. In aspects, the botulism is caused by Type A Botulinum toxin, Type B Botulinum toxin, Type C1 Botulinum toxin, Type C2 Botulinum toxin, Type D Botulinum toxin, Type F Botulinum toxin or Type G Botulinum toxin. In aspects, the composition also includes an antitoxin and/or an antibiotic.

Embodiments include methods of treating a patient with botulism. The methods can include administration of a therapeutic amount of Type E Botulinum toxin.

In embodiments, the botulism is caused by Type A Botulinum toxin and causes one or more of dysphagia, difficulty breathing, muscle weakness, double vision, drooping of eyelids, loss of facial expression, dry mouth and throat, postural hypotension, constipation, nausea, vomiting and difficulty with talking.

In embodiments, the botulism is infant botulism or wound botulism. Alternatively, the botulism is the result of foodborne botulism or results from therapeutic use of Type A Botulinum toxin or Type B Botulinum toxin. In aspects, the botulism results from therapeutic or cosmetic use of botulinum toxin.

In embodiments, the methods also include administration of an antitoxin and/or an antibiotic to the subject.

In embodiments, the BoNT/E is administered at multiple injection sites. Each injection can be, for example, a volume of about 0.2 ml or less. In embodiments, a total of 0.01 mg to 0.5 mg of BoNT/E in solution is administered by injection. In embodiments, a total of 0.02 mg to 0.05 mg of BoNT/E is administered. In embodiments, 0.1-1 ng/kg of BoNT/E is administered. The BoNT/E can be administered by injection using a syringe with a rack and pinion component.

Embodiments also include a method of treating botulism in a patient that includes steps of (a) identifying a patient with signs and/or symptoms of botulism, (b) treating the patient by administering an effective amount of Type E Botulinum toxin to the subject, (c) monitoring signs and/or symptoms of botulism and (d) repeating steps b) and c) until the signs and/or symptoms of botulism subside. The method can also include a step of identifying the serotype of botulism. The method can include a step of confirming the presence of botulism (i.e., diagnosis by signs/symptoms and/or by identifying the toxin or bacteria in the patient).

In embodiments, the botulism is the result of exposure to one or more of Type A Botulinum toxin, Type B Botulinum toxin, Type C1 Botulinum toxin, Type C2 Botulinum toxin, Type D Botulinum toxin, Type F Botulinum toxin or Type G Botulinum toxin.

The treatment of botulism can include multiple injections at a plurality of sites. Each injection can be a volume of about 0.2 ml or less. The BoNT/E can be injected using a syringe with a rack and pinion component which allows accurate and precise injection of small volumes. When injected, the volumes (and activity) are generally smaller (e.g., 100 to 7,500 units). Alternatively, the BoNT/E can be administered intravenously. The total amount of BoNT/E administered intravenously can be about 1,000 to 20,000 Units. Alternatively, the therapeutic amount of Type E Botulinum toxin is about 1,500 to 30,000 Units or about 2,000 to 40,000 Units.

Embodiments also include a method of increasing the rate of turnover of the synapses at the neuromuscular junction by administering Type E Botulinum toxin (BoNT/E). In embodiments, the BoNT/E increases the rate of sprouting of cholinergic nerve terminals.

Embodiments also include a pharmaceutical composition that includes Type E Botulinum toxin for use in the treatment of botulism. The botulism can be Type A Botulinum toxin, Type B Botulinum toxin, Type C1 Botulinum toxin, Type C2 Botulinum toxin, Type D Botulinum toxin, Type F Botulinum toxin or Type G Botulinum toxin.

### BRIEF DESCRIPTION OF THE FIGURES

FIG. 1 is a graphical depiction of the cleavage sites of the main types of Botulinum toxin (BoNT/A, BoNT/B, BoNT/C, BoNT/D, BoNT/E, BoNT/G and BoNT/G) and tetanus neurotoxin (TeNT) at the neuromuscular junction.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “Accufill syringe” refers to a syringe with a rack and pinion component. This is in contrast to a conventional syringe that includes a plunger and a barrel. An Accufill syringe can be preferred for injection because scar tissue is dense and higher pressure may be necessary to inject the drug product. The design is also conducive to continuous flow with precise delivery. The rack and pinion assembly has two components (i.e., a toothed rack and a pinion gear).

The term “Clostridium botulinum” or C. botulinum refers to a Gram-positive, rod-shaped, spore-forming bacterium. It is an obligate anaerobe, meaning that oxygen is poisonous to the cells. However, C. botulinum tolerates traces of oxygen due to the enzyme superoxide dismutase, which is an important antioxidant defense in nearly all cells exposed to oxygen. C. botulinum is able to produce the neurotoxin only during sporulation, which can happen only in an anaerobic environment. C. botulinum is divided into four distinct phenotypic groups (I-IV) and is also classified into seven serotypes (A-G) based on the antigenicity of the botulinum toxin produced.

Botulism infection most often occurs in one of five different ways:
Foodborne botulism: When a person ingests the preformed toxin.
Infant botulism: This occurs in a small population of infants (i.e., children under the age of one year) who have C. botulinum colonization in their gastrointestinal tract.
Wound botulism: When infected wounds contain C. botulinum, and they secrete the toxin.
Iatrogenic botulism: This occurs when cosmetic or therapeutic procedures that use the C. botulinum toxin cause systemic intoxication.
Intestinal colonization: When a person over the age of one year harbors the C. botulinum toxin within the gastrointestinal tract (infantile botulism in children and adults).
   Even though there are multiple ways to contact botulism, only three main serotypes are responsible for these infections:
Type A: Predominantly found west of the Mississippi River
Type B: Predominantly found east of the Mississippi River
Type E: Found in the Pacific Northwest with a preponderance in Alaska

The term “Botulinum toxin serotype” or “BoNT serotype” refers to one of the standard known BoNT serotypes (e.g., BoNT/A, BoNT/C, BoNT/D, BoNT/E, BoNT/F, etc.). BoNT serotypes differ from each other by as little as about 35% at the amino acid level (e.g., between BoNT/E and BoNT/F) up to about 66% at the amino acid level, (e.g., for BoNT/A vs BoNT/C or D). Thus, BoNT serotypes differ from each other by about 35-66% at the amino acid level. The term “BoNT subtype” (e.g., a BoNT/A1A subtype) refers to botulinum neurotoxin gene sequences of a particular serotype (e.g., A, C, D, F, etc.) that differ from each other sufficiently to produce differential antibody binding.

Botulinum neurotoxin type A is the potent disease agent in botulism. Botulinum neurotoxin type B is used for treatment of severe spasms in the neck muscles. Botulinum neurotoxin type C induces apoptotic cell death in cerebellar neurons acting in birds, mammals and fish. Botulinum neurotoxin type D inhibits the release of TNF from monocytes. Botulinum neurotoxin type D/C binds and enters efficiently into neurons lacking complex gangliosides. It recognizes only sialic acid but no other gangliosides' moieties. Botulinum neurotoxin type E, F and G act on human proteins of the neuroexocytosis apparatus. Botulinum neurotoxin type X cleaves both canonical and non-canonical vesicle-associated membrane proteins.

Botulism can be local and/or systemic. An infection that affects only one body part or organ is referred to as a localized infection. For example, wound botulism can be confined to a specific area of the body. An infection that is in the bloodstream is referred to as a systemic infection. For example, Iatrogenic botulism can lead to systemic intoxication after cosmetic or therapeutic procedures.

The term “parenteral drug administration” refers to any non-oral means of administration but is generally interpreted as relating to injecting directly into the body, bypassing the skin and mucous membranes. Accordingly, parental administration can be intravenous or subcutaneous.

The term “neutralization” refers to a measurable decrease in the toxicity of a Botulinum neurotoxin (e.g., BoNT/A). Reduced toxicity can be measured as an increase in the time that paralysis developed and/or as a lethal dosage (e.g., LD50).

The term “antitoxin” refers to an antibody with the ability to neutralize a specific toxin. As used herein, the toxin is one or more serotypes of Clostridium botulinum neurotoxin (BoNT).

The term “SNARE protein” or “SNAP receptor protein” refers to a large protein family that includes more than 60 members in mammalian cells. The primary role of SNARE proteins is to mediate vesicle fusion (i.e., the fusion of vesicles with the target membrane). This mediates exocytosis and can also mediate the fusion of vesicles with membrane-bound compartments (e.g., a lysosome). The best studied SNAREs are those that mediate the neurotransmitter release of synaptic vesicles in neurons. These neuronal SNAREs are the targets of the neurotoxins responsible for botulism and tetanus produced by certain bacteria.

SNAP-25 proteins are the targets of the neurotoxins responsible for botulism and tetanus produced by certain bacteria. SNAP-25 is a component of the SNARE complex, which is central to synaptic vesicle exocytosis, and, by directly interacting with different calcium channels subunits, it negatively modulates neuronal voltage-gated calcium channels, thus regulating intracellular calcium dynamics.

Botulinum Toxin (BoNT) cleaves SNARE proteins in neurons. The action of BoNT follows a four-step mechanism that includes (1) binding to the neuronal membrane, (2) endocytosis, (3) membrane translocation and (4) proteolysis of SNARE proteins.

The term “activity” refers to the activity of BoNT as measured by its “LD” or “Lethal Dose.” The LD₅₀ is the amount of a material, given all at once, which causes the death of half of a group of test animals. The LD₅₀ is one way to measure the short-term poisoning potential (i.e., acute toxicity) of a substance. The LD₅₀ of Botulinum toxin has been determined across several animal species but not in humans. A unit of BoNT-A usually is defined in terms of its biologic potency. One mouse unit (MU) of BoNT-A equals the LD₅₀ for a 20-g Swiss-Webster mouse. However, BoNT-A sensitivity varies among different species. The LD₅₀ in monkeys has been determined as 39 U/kg. Based on these findings from primate studies, human LD₅₀ is estimated at approximately 3000 U for a 70 kg adult. Typical doses of BOTOX for larger muscle groups range from 60-400 total units given in a single treatment; however, because of inadequate understanding of the complete dose response curve in humans, a relative ceiling dose of 360 U, given no sooner than 12 weeks apart, is generally recommended.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e. arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e. reversing the pathology and/or symptomatology) such as decreasing the severity of disease.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Botulinum toxin produced by Clostridium botulinum is the cause of botulism. Humans most commonly ingest the toxin from eating improperly canned foods in which C. botulinum has grown. However, the toxin can also be introduced through an infected wound. In infants, the bacteria can sometimes grow in the intestines and produce botulinum toxin within the intestine and can cause a condition known as “floppy baby syndrome.” In all cases, the toxin can then spread, blocking nerves and muscle function. In severe cases, the toxin can block nerves controlling the respiratory system or heart leading to death. Botulism can be difficult to diagnose, as it may appear similar to diseases such as Guillain-Barré syndrome, myasthenia gravis and stroke.

Conventional treatments of botulism include administration of antitoxin and intensive palliative care of the patient. The only specific strategy to treat botulism is to neutralize the circulating toxin with an antitoxin, thus preventing the irreversible internalization of BoNT into the neurons. The antitoxin product available for treatment of botulism in infants is a human-derived immune globulin, named BabyBIG. To treat botulism in non-infant patients, an equine-derived heptavalent botulinum antitoxin (HBAT) may be available. However, animal-derived antitoxin treatment can cause side effects ranging from local skin reactions to serum sickness.

### Botox-Related Adverse Events

Botulism can also result from therapeutic administration of Botox. There are two classes of Botox-related adverse events—transient/benign events, and potentially serious events. Benign side effects are well-localized, reversible and self-limited complications which develop within a few days of the injection, and they usually disappear without any treatment. The aesthetic and functional adverse effects are associated with different muscle responses to botulinum toxin or with misplacement of botulinum toxin. The serious events are sequelae due to the systemic spread of toxin leading to botulism.

In addition to injection site complications, there is a group of aesthetic and functional adverse effects associated with different muscle responses to botulinum toxin or with the misplacement of botulinum toxin. They are specific for botulinum toxin and are an effect of the direct pharmacological action of the toxin. They are primarily caused by temporary denervation of adjacent muscles outside of the intended treatment area. These complications are technique-dependent, because they are usually due to misplacement of the toxin or to different muscle reactivity. When adjacent muscles are inadvertently paralyzed, both aesthetic and functional problems may arise. There are some well-known, mild side effects of botulinum toxin treatment on the upper face. Most are self-limiting with time. Common adverse effects can occur due to percutaneous injections on the forehead. The most common complication in the treatment of the glabellar complex is ptosis of the upper eyelid known as blepharoptosis.

Potentially serious events include sequelae due to the systemic spread of toxin leading to botulism-like features or systemic anaphylactic reactions. The US Food and Drug Administration (FDA) describes adverse effects as serious if they meet the strict criteria. They are classified as a life-threatening experience requiring intervention to prevent permanent damage and associated with patient hospitalization or prolongation of existing hospitalization, and persistent or significant disability. In 2005, the FDA reported 36 adverse effects related to cosmetic use, which met the FDA's definition of serious adverse events, with the majority concerning dysphagia. Serious adverse effects after cosmetic use of botulinum toxin include dysphagia, muscle weakness, and allergic reactions.

### BoNT Mechanism of Action

Botulinum Toxin (BoNT) cleaves SNARE proteins in neurons. The action of BoNT follows a four-step mechanism including (1) binding to the neuronal membrane, (2) endocytosis, (3) membrane translocation and (4) proteolysis of SNARE proteins.

In its mechanism of action, the heavy chain of BoNT is first used to find its neuronal targets and bind to the gangliosides and membrane proteins of presynaptic neurons. Next, the toxin is then endocytosed into the cell membrane. The heavy chain undergoes a conformational change important for translocating the light chain into the cytosol of the neuron. Finally, after the light chain of BoNT is brought into the cytosol of the targeted neuron, it is released from the heavy chain so that it can reach its active cleavage sites on the SNARE proteins. The light chain is released from the heavy chain by the reduction of the disulfide bond holding the two together. The reduction of this disulfide bond is mediated by the NADPH-thioredoxin reductase-thioredoxin system. The light chain of BoNT acts as a metalloprotease on SNARE proteins that is dependent on Zn(II) ions, cleaving them and eliminating their function in exocytosis.

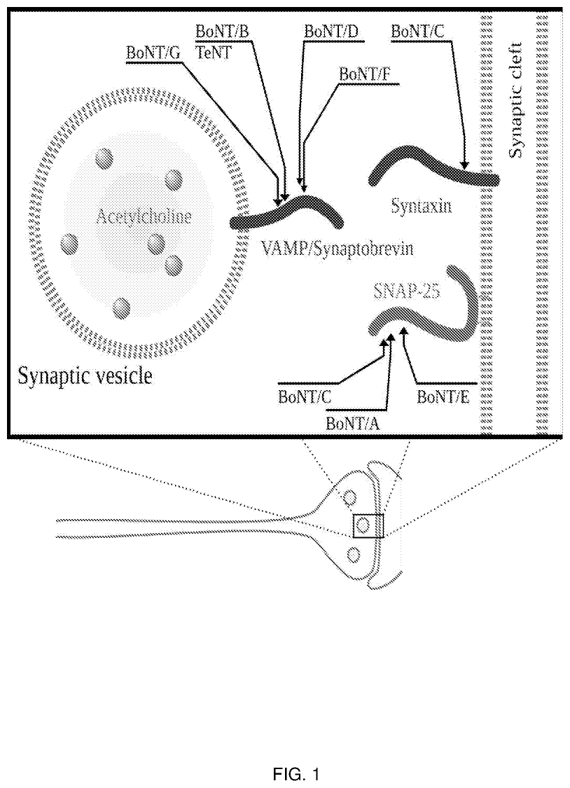

There are eight known isotypes of BoNT, BoNT/A-BoNT/H, each with different specific cleavage sites on SNARE proteins. SNAP25, a member of the SNARE protein family located in the membrane of cells, is cleaved by BoNT isotypes A, C, and E. The cleavage of SNAP-25 by these isotypes of BoNT greatly inhibits their function in forming the SNARE complex for fusion of vesicles to the synaptic membrane. BoNT/C also targets Syntaxin-1, another SNARE protein located in the synaptic membrane. It degenerates these Syntaxin proteins with a similar outcome as with SNAP-25. A third SNARE protein, Synaptobrevin (VAMP), is located on cell vesicles. VAMP2 is targeted and cleaved by BoNT isotypes B, D, and F in synaptic neurons. The targets of these isotypes of BoNT are depicted in FIG. 1.

In each of these cases, Botulinum Neurotoxin causes functional damage to SNARE proteins, which has significant physiological and medical implications. By damaging SNARE proteins, the toxin prevents synaptic vesicles from fusing to the synaptic membrane and releasing their neurotransmitters into the synaptic cleft. With the inhibition of neurotransmitter release into the synaptic cleft, action potentials cannot be propagated to stimulate muscle cells. This results in paralysis of those infected and in serious cases it can cause death. Although the effects of botulinum neurotoxin can be fatal, it has also been used as a therapeutic agent in medical and cosmetic treatments.

### Methods of Treatment Using Type E Botulinum Toxin

Applicant has found that the use of Botulism from Type E Botulinum toxin (BoNT/E) can be used therapeutically to treat Botulism caused by one of the other serotypes of botulinum toxin (e.g., A, B, C1, C2, D, F and G). For example, BoNT/E can be administered (e.g. parentally) to a subject suffering from infection of Type A Botulinum toxin (BoNT-A) or Type C Botulinum toxin (BoNT-C).

Upon internalization into the neuronal cytosol, BoNTs exert their toxic effect by cleaving one of three soluble N-ethylmaleimide-sensitive factor attachment protein receptor (SNARE) proteins that are integral to vesicular trafficking and neurotransmitter release. The specific SNARE protein targeted and the site of hydrolytic cleavage vary among the seven BoNT serotypes. BoNT serotypes A and E specifically cleave SNAP-25 at a unique peptide bond. BoNT serotypes B, D, F, and G hydrolyze VAMP/synaptobrevin, at different single peptide bonds, and BoNT/C cleaves both syntaxin and SNAP-25

Without being bound by theory, Applicant proposes the use of Type E Botulinum toxin leads to a faster physiologic response mechanism to repair or replace the SNAP Protein. Type A and Type C Botulinum toxin cleave SNAP Protein close to the terminal portion (i.e., within about 25 amino acids). This cleavage is significant in that it renders the protein ineffective. In contrast, Type E Botulinum toxin cleaves SNAP Protein further away from the terminal portion (i.e., within a few hundred amino acids). This also renders the protein ineffective. However, because it is a more significant change to the protein, the physiological repair mechanism is activated more quickly. Thus, the shorter version of SNAP protein (as cleaved by BoNT/E) can speed the healing/repair process.

As shown in FIG. 1, Type A Botulinum toxin cleaves SNAP protein close to the end. It generally takes a few months for repair. In contrast, Type E Botulinum toxin cleaves SNAP protein further away from end of the molecule in which case, the repair process is faster (i.e., one to three weeks). Moreover, because of the different cleavage sites, botulism patients will not experience exasperated symptoms with exposure to Type E Botulinum. This is because the SNAP-25 protein has already been inactivated (i.e., by BoNT-A). Further proteolysis of SNAP-25 protein can speed the healing/repair process without increasing the extent or severity of symptoms.

In embodiments, the BoNT/E is administered with one or more additional agents such as an antitoxin or antibiotic. The antibiotic can be, for example, penicillin, streptomycin, and amoxicillin, although combinations of the same may be preferred. The antibiotic can be administered by injection into the muscle, although the skilled person may select the most appropriate route of administration, depending on the particular antibiotic. Without being bound by theory, it is thought that penicillin functions as an antibiotic by interfering with the cell wall synthesis of bacteria, leading to rupture of the cell. Although it may be effective in the destruction of Clostridium botulinum cells, it is not thought to react in any way with the neurotoxins produced by this organism and should have no discernable effects on the development of botulism.

Doses of the antibiotics will be those prescribed routinely. The amount of the antibiotic administered may be selected by one skilled in the art. Optionally, the antibiotic is administered in an amount of 7.5-12.5 milligrams (mg) per kilogram body weight of the subject. Alternatively, penicillin and streptomycin can be administered in a total amount 7.5-12.5 mg per kilogram body weight of the subject and amoxicillin is administered in an amount of 7.5-12.5 mg per kilogram body weight of the animal, optionally daily.

### Utility and Administration

For use as treatment of human and animal subjects, the compounds of the invention can be formulated as pharmaceutical or veterinary compositions. Depending on the subject to be treated, the mode of administration, and the type of treatment desired (e.g., prevention, prophylaxis, or therapy) the compounds are formulated in ways consonant with these parameters. A summary of such techniques can be found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

In one embodiment, the Type E Botulinum toxin (BoNT/E) drug product is stored and shipped in sterile lyophile (e.g., 3 mL clear Type I glass vial with a chlorobutyl rubber stopper with no latex) and capped with an overseal and blue tear-off cap or a 2-mL clear Type I glass vial with a chlorobutyl rubber stopper (no latex) and capped with an overseal and red tear-off cap. The cake is reconstituted before subject use with single-use sterile diluent (2 mM calcium chloride and 154 mM sodium chloride solution in water for injection or 2 mM calcium chloride and 103 mM sodium chloride in water for injection).

In one embodiment, sterile diluent for reconstitution of BoNT/E is provided in a 5 mL or a 2 mL clear Type I glass vial with a chlorobutyl rubber stopper (no latex) and capped with an overseal and blue tear-off cap. A suitable sterile buffered diluent can include 10 mM Tris, 2 mM CaCl, and 154 mM NaCl.

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### Administration

A pharmaceutical composition comprising BoNT/E in accordance with the present disclosure can be formulated in any pharmaceutically acceptable carrier(s) or excipient(s). As used herein, the term “pharmaceutically acceptable carrier” includes any and all solvents, dispersion media, coatings, antibacterial and antifungal agents, isotonic and absorption delaying agents, and the like that are physiologically compatible. Pharmaceutical compositions can include suitable solid or gel phase carriers or excipients. Exemplary carriers or excipients include calcium carbonate, calcium phosphate, various sugars, starches, cellulose derivatives, gelatin, and polymers such as polyethylene glycols. Exemplary pharmaceutically acceptable carriers include one or more of water, saline, phosphate buffered saline, dextrose, glycerol, ethanol and the like, as well as combinations thereof. In many cases it will be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the therapeutic agents.

The BoNT/E can be incorporated into a pharmaceutical composition suitable for parenteral administration. Suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05%>polysorbate-80 (optimally 0.005-0.01%). Additional surfactants include but are not limited to polysorbate 20 and BRIJ surfactants.

The BoNT/E preparation can be lyophilized and stored as sterile powders, preferably under vacuum, and then reconstituted in bacteriostatic water (containing, for example, benzyl alcohol preservative) or in sterile water prior to injection. Pharmaceutical compositions can be formulated for parenteral administration by injection e.g., by bolus injection or continuous infusion.

The therapeutic agents in the pharmaceutical compositions can be formulated in a “therapeutically effective amount” or a “prophylactically effective amount”. A “therapeutically effective amount” refers to an amount effective, at dosages and for periods of time necessary, to achieve the desired therapeutic result. A therapeutically effective amount of the protease (i.e. BONT/E) may vary depending on the condition to be treated, the severity and course of the condition, the mode of administration, whether the agent is administered for preventive or therapeutic purposes, the bioavailability of the particular agent, the ability of the agent to elicit a desired response in the individual, previous therapy, the age, weight and sex of the patient, the patient's clinical history and response to the agent, discretion of the attending physician, etc. A therapeutically effective amount is also one in which any toxic or detrimental effects are not outweighed by the therapeutically beneficial effects. A “prophylactically effective amount” refers to an amount effective, at dosages and for periods of time necessary, to achieve the desired prophylactic result.

The solution containing BoNT/E is suitably administered to the patient at one time or over a series of treatments and may be administered to the patient at any time from diagnosis onwards. The solution can be administered as the sole treatment or in conjunction with other drugs or therapies useful in treating the condition in question.

As a general proposition, a therapeutically effective amount or prophylactically effective amount of BoNT/E will be administered in a range from about 1 ng/kg body weight to about 100 mg/kg body weight whether by one or more administrations. In a particular embodiment, the agent is administered in the range of from about 1 ng/kg body weight to about 10 mg/kg body weight, about 1 ng/kg body weight to about 1 mg/kg body weight, about 1 ng/kg body weight to about 100 g/kg body weight, about 1 ng/kg body weight to about 10 g/kg body weight, about 1 ng/kg body weight/day to about 1 g/kg body weight, about 1 ng/kg body weight to about 100 ng/kg body weight, about 1 ng/kg body weight to about 10 ng/kg body weight, about 10 ng/kg body weight to about 100 mg/kg body weight, about 10 ng/kg body weight to about 10 mg/kg body weight, about 10 ng/kg body weight to about 1 mg/kg body weight, about 10 ng/kg body weight/to about 100 g/kg body weight, about 10 ng/kg body weight to about mg/kg body weight, about 10 ng/kg body weight to about 1 mg/kg body weight, 10 ng/kg body weight to about 100 ng/kg body weight, about 100 ng/kg body weight to about 100 mg/kg body weight, about 100 ng/kg body weight to about 10 mg/kg body weight, about 100 ng/kg body weight to about 1 mg/kg body weight, about 100 ng/kg body weight to about 100 mg/kg body weight, about 100 ng/kg body weight to about 10 mg/kg body weight, about 100 ng/kg body weight to about 1 mg/kg body weight, about 1 mg/kg body weight to about 100 mg/kg body weight, about 1 mg/kg body weight to about 10 mg/kg body weight/day, about 1 mg/kg body weight to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight, about 10 mg/kg body weight to about 10 mg/kg body weight, about mg/kg body weight to about 1 mg/kg body weight/day, about 10 mg/kg body weight to about 100 mg/kg body weight, about 100 mg/kg body weight/day to about 100 mg/kg body weight, about 100 mg/kg body weight/day to about 10 mg/kg body weight, about 100 mg/kg body weight/day to about 1 mg/kg body weight, about 1 mg/kg body weight to about 100 mg/kg body weight, about 1 mg/kg body weight to about 10 mg/kg body weight, about 10 mg/kg body weight to about 100 mg/kg body weight/day.

In other embodiments, BoNT/E is administered in the range of about 10 ng to about 100 ng per individual administration, about 10 ng to about 1 g per individual administration, about 10 ng to about 10 g per individual administration, about 10 ng to about 100 mg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 mg per individual administration, about 100 ng to about 10 mg per individual administration, about 100 ng to about 100 mg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 mg to about 10 mg per individual administration, about 1 mg to about 100 mg per individual administration, about 1 mg to about 1000 mg per individual administration, about 1 mg to about 10,000 mg per individual administration, about 10 mg to about 100 mg per individual administration, about 10 mg to about 1000 mg per individual administration, about 10 mg to about 10,000 mg per individual administration, about 100 mg to about 1000 mg per individual administration, about 100 mg to about 10,000 mg per individual administration, The agent may be administered daily, twice daily, thrice daily, four times daily, five times daily, every 2, 3, 4, 5, 6 or 7 days, or every 1, 2, 3 or 4 weeks.

In yet other embodiments, BoNT/E is administered in the range of about 10 ng to about 100 ng per injection, about 10 ng to about 1 g per injection, about 10 ng to about 10 g per injection, about 10 ng to about 100 mg per injection, about 10 ng to about 1 mg per injection, about 10 ng to about 10 mg per injection, about 10 ng to about 100 mg per injection, about 10 ng to about 1000 mg per injection, about 10 ng to about mg per injection, about 100 ng to about 1 mg per injection, about 100 ng to about 10 mg per injection, about 100 ng to about 100 mg per injection, about 100 ng to about 1 mg per injection, about 100 ng to about 10 mg per injection, about 100 ng to about 100 mg per injection, about 100 ng to about 1000 mg per injection, about 100 ng to about 10,000 mg per injection, about 1 mg to about 10 mg per injection, about 1 mg to about 100 mg per injection, about 1 mg to about 1000 mg per injection, about 1 mg to about 10,000 mg per injection, about 10 mg to about 100 mg per injection, about 10 mg to about 1000 mg per injection, about 10 mg to about 10,000 mg per injection, about 100 mg to about 1000 mg per injection, about 100 mg to about 10,000 mg per injection, The agent may be injected daily, twice daily, thrice daily, four times daily, five times daily, every 2, 3, 4, 5, 6 or 7 days, or every 1, 2, 3 or 4 weeks.

In other particular embodiments, the amount of the BoNT/E can be administered at a dose of about 0.0006 mg, 0.001 mg, 0.003 mg, 0.006 mg, 0.01 mg, mg, 0.06 mg, 0.1 mg, 0.3 mg, 0.6 mg, 1 mg, 3 mg, 6 mg, 10 mg, 30 mg, 60 mg, 100 mg, 300 mg, 600 mg, 1000 mg, 2000 mg, 5000 mg or 10,000 mg. As expected, the dosage will be dependent on the condition, size, age and condition of the patient.

In embodiments, the amount of the BoNT/E is administered intravenously at a therapeutically effective of a range from about 1,000-10,000 units, 10,000-50,000 units, 10,000-100,000 units, 20,000-50,000 units, 20,000-100,000 units, 50,000-100,000 units, 5,000-15,000 units, 5,000-20,000 units, 5,000-25,000 units, 5,000-units, 10,000-20,000 units, 10,000-25,000 units, 10,000-30,000 units, 35,000 units, 10,000-40,000 units, 20,000-25,000 units, 20,000-30,000 units, 20,000-35,000 units, 20,000-40,000 units, 30,000-40,000 units, 10,000-units, 30,000-50,000 units, or 40,000-50,000 units per treatment session.

In embodiments, the amount of the BoNT/E is administered by injection at a therapeutically effective of a range from about 1-10 units, 10-50 units, 10-100 units, 20-50 units, 20-100 units, 50-100 units, 50-150 units, 50-200 units, 50-250 units, 50-300 units, 100-200 units, 100-250 units, 100-300 units, 100-350 units, 100-400 units, 200-250 units, 200-300 units, 200-350 units, 200-400 units, 300-400 units, 100-400 units, 300-500 units, or 400-500 units per treatment session.

In other aspects of this embodiment, a pharmaceutical composition disclosed herein reduces the signs/symptoms of botulism by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein reduces the signs/symptoms of botulism from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In other aspects of this embodiment, a pharmaceutical composition disclosed herein improves the signs/symptoms of botulism by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein improves the signs/symptoms of botulism from, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, about 60% to about 80%, about 10% to about 70%, about 20% to about 70%, about 30% to about 70%, about 40% to about 70%, or about 50% to about 70%.

In other aspects of this embodiment, a pharmaceutical composition disclosed herein reduces the size of a region of prostate tissue by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In yet other aspects of this embodiment, a pharmaceutical composition disclosed herein reduces the size of a region of prostate tissue by, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A pharmaceutical composition disclosed herein is in an amount sufficient to allow customary administration to an individual. In aspects of this embodiment, a pharmaceutical composition disclosed herein can be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition. In other aspects of this embodiment, a pharmaceutical composition disclosed herein may be, e.g., at least 5 mg, at least 10 mg, at least 20 mg, at least 25 mg, at least 50 mg, at least 75 mg, at least 100 mg, at least 200 mg, at least 300 mg, at least 400 mg, at least 500 mg, at least 600 mg, at least 700 mg, at least 800 mg, at least 900 mg, at least 1,000 mg, at least 1,100 mg, at least 1,200 mg, at least 1,300 mg, at least 1,400 mg, or at least 1,500 mg of a pharmaceutical composition. In yet other aspects of this embodiment, a pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, or about 1,000 mg to about 1,500 mg. In still other aspects of this embodiment, a pharmaceutical composition disclosed herein may be in the range of, e.g., about 10 mg to about 250 mg, about 10 mg to about 500 mg, about 10 mg to about 750 mg, about 10 mg to about 1,000 mg, about 10 mg to about 1,500 mg, about 50 mg to about 250 mg, about 50 mg to about 500 mg, about 50 mg to about 750 mg, about 50 mg to about 1,000 mg, about 50 mg to about 1,500 mg, about 100 mg to about 250 mg, about 100 mg to about 500 mg, about 100 mg to about 750 mg, about 100 mg to about 1,000 mg, about 100 mg to about 1,500 mg, about 200 mg to about 500 mg, about 200 mg to about 750 mg, about 200 mg to about 1,000 mg, about 200 mg to about 1,500 mg, about 5 mg to about 1,500 mg, about 5 mg to about 1,000 mg, or about 5 mg to about 250 mg.

A pharmaceutical composition disclosed herein can include a solvent, emulsion or other diluent in an amount sufficient to dissolve a pharmaceutical composition disclosed herein. In other aspects of this embodiment, a pharmaceutical composition disclosed herein may comprise a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects of this embodiment, a pharmaceutical composition disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

The final concentration of a pharmaceutical composition disclosed herein can be of any concentration desired. In an aspect of this embodiment, the final concentration of a pharmaceutical composition may be a therapeutically effective amount. In other aspects, the final concentration of a pharmaceutical composition may be, e.g., at least 0.00001 mg/mL, at least 0.0001 mg/mL, at least 0.001 mg/mL, at least mg/mL, at least 0.1 mg/mL, at least 1 mg/mL, at least 10 mg/mL, at least 25 mg/mL, at least 50 mg/mL, at least 100 mg/mL, at least 200 mg/mL or at least 500 mg/mL. In other aspects of this embodiment, the final concentration of a pharmaceutical composition may be in a range of, e.g., about 0.00001 mg/mL to about 3,000 mg/mL, about 0.0001 mg/mL to about 3,000 mg/mL, about 0.01 mg/mL to about 3,000 mg/mL, about 0.1 mg/mL to about 3,000 mg/mL, about 1 mg/mL to about 3,000 mg/mL, about 250 mg/mL to about 3,000 mg/mL, about 500 mg/mL to about 3,000 mg/mL, about 750 mg/mL to about 3,000 mg/mL, about 1,000 mg/mL to about 3,000 mg/mL, about 100 mg/mL to about 2,000 mg/mL, about 250 mg/mL to about 2,000 mg/mL, about 500 mg/mL to about 2,000 mg/mL, about 750 mg/mL to about 2,000 mg/mL, about 1,000 mg/mL to about 2,000 mg/mL, about 100 mg/mL to about 1,500 mg/mL, about 250 mg/mL to about 1,500 mg/mL, about 500 mg/mL to about 1,500 mg/mL, about 750 mg/mL to about 1,500 mg/mL, about 1,000 mg/mL to about 1,500 mg/mL, about 100 mg/mL to about 1,200 mg/mL, about 250 mg/mL to about 1,200 mg/mL, about 500 mg/mL to about 1,200 mg/mL, about 750 mg/mL to about 1,200 mg/mL, about 1,000 mg/mL to about 1,200 mg/mL, about 100 mg/mL to about 1,000 mg/mL, about 250 mg/mL to about 1,000 mg/mL, about 500 mg/mL to about 1,000 mg/mL, about 750 mg/mL to about 1,000 mg/mL, about 100 mg/mL to about 750 mg/mL, about 250 mg/mL to about 750 mg/mL, about 500 mg/mL to about 750 mg/mL, about 100 mg/mL to about 500 mg/mL, about 250 mg/mL to about 500 mg/mL, about mg/mL to about 0.0001 mg/mL, about 0.00001 mg/mL to about 0.001 mg/mL, about 0.00001 mg/mL to about 0.01 mg/mL, about 0.00001 mg/mL to about 0.1 mg/mL, about 0.00001 mg/mL to about 1 mg/mL, about 0.001 mg/mL to about 0.01 mg/mL, about 0.001 mg/mL to about 0.1 mg/mL, about 0.001 mg/mL to about 1 mg/mL, about mg/mL to about 10 mg/mL, or about 0.001 mg/mL to about 100 mg/mL.

Aspects of the present specification disclose, in part, treating an individual suffering from botulism. As used herein, the term “treating,” refers to reducing or eliminating in an individual a clinical symptom of botulism; or delaying or preventing in an individual the onset of a clinical symptom of botulism. For example, the term “treating” can mean reducing signs/symptoms of botulism, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with botulism are well known and can be determined by a person of ordinary skill in the art. Those of skill in the art will know the appropriate symptoms or indicators associated with a specific type of ailment and will know how to determine if an individual is a candidate for treatment as disclosed herein.

In another aspect, a pharmaceutical composition disclosed herein reduces the severity of a symptom of a disorder associated with botulism. In aspects of this embodiment, a pharmaceutical composition disclosed herein reduces the severity of a symptom of a disorder associated with botulism by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95%. In other aspects of this embodiment, a pharmaceutical composition disclosed herein reduces the severity of a symptom of a disorder associated with botulism by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In aspects of this embodiment, a therapeutically effective amount of a pharmaceutical composition disclosed herein reduces a symptom associated with botulism or reduces the time of recovery by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces a symptom associated with botulism or reduces the time of recovery by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition disclosed herein reduces a symptom associated with botulism or reduces the time of recovery by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In yet other aspects of this embodiment, a therapeutically effective amount of a pharmaceutical composition disclosed herein generally is in the range of about 0.001 mg/kg to about 100 mg/kg and administered, for example, every 3, 5, 7, 10 or 14 days. In aspects of this embodiment, an effective amount of a pharmaceutical composition disclosed herein may be, e.g., at least 0.001 mg/kg, at least 0.01 mg/kg, at least 0.1 mg/kg, at least 1.0 mg/kg, at least 5.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, or at least 50 mg/kg and administered, for example, every 3, 7, 10 or 14 days. In other aspects of this embodiment, an effective amount of a pharmaceutical composition disclosed herein may be in the range of, e.g., about 0.001 mg/kg to about 10 mg/kg, about 0.001 mg/kg/day to about 15 mg/kg, about 0.001 mg/kg to about 20 mg/kg, about 0.001 mg/kg to about 25 mg/kg, about 0.001 mg/kg to about mg/kg, about 0.001 mg/kg to about 35 mg/kg, about 0.001 mg/kg to about 40 mg/kg, about 0.001 mg/kg to about 45 mg/kg, about 0.001 mg/kg to about 50 mg/kg, about mg/kg to about 75 mg/kg, or about 0.001 mg/kg to about 100 mg/kg and administered, for example, every 3, 5, 7, 10 or 14 days. In yet other aspects of this embodiment, an effective amount of a pharmaceutical composition disclosed herein may be in the range of, e.g., about 0.01 mg/kg to about 10 mg/kg, about 0.01 mg/kg to about 15 mg/kg, about 0.01 mg/kg to about 20 mg/kg, about 0.01 mg/kg to about 25 mg/kg, about 0.01 mg/kg to about 30 mg/kg, about 0.01 mg/kg to about 35 mg/kg, about mg/kg to about 40 mg/kg, about 0.01 mg/kg to about 45 mg/kg, about 0.01 mg/kg to about 50 mg/kg, about 0.01 mg/kg to about 75 mg/kg, or about 0.01 mg/kg to about 100 mg/kg and administered, for example, every 3, 5, 7, 10 or 14 days. In still other aspects of this embodiment, an effective amount of a pharmaceutical composition disclosed herein may be in the range of, e.g., about 0.1 mg/kg to about 10 mg/kg, about mg/kg to about 15 mg/kg, about 0.1 mg/kg to about 20 mg/kg, about 0.1 mg/kg to about 25 mg/kg, about 0.1 mg/kg to about 30 mg/kg, about 0.1 mg/kg to about 35 mg/kg, about 0.1 mg/kg to about 40 mg/kg, about 0.1 mg/kg to about 45 mg/kg, about mg/kg to about 50 mg/kg, about 0.1 mg/kg to about 75 mg/kg, or about 0.1 mg/kg to about 100 mg/kg and administered, for example, every 3, 5, 7, 10 or 14 days.

Dosing can be single dosage or cumulative (serial dosing), and can be readily determined by one skilled in the art. For example, treatment of botulism can include a one-time administration of an effective dose of a pharmaceutical composition disclosed herein. Alternatively, treatment of botulism can include multiple administrations of an effective dose of a pharmaceutical composition carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, a therapeutic disclosed herein is capable of reducing the signs/symptoms of botulism in an individual suffering from the disease or reducing the time of recovery by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, the signs/symptoms of botulism in an individual suffering from the disease or the time of recovery are reduced by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In one embodiment, a therapeutic disclosed herein is capable of reducing the signs/symptoms of botulism in an individual suffering from a botulism by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, a therapeutic disclosed herein is capable of reducing the recovery time (i.e., healing time) in an individual by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In an embodiment, the period of administration of a BoNT/E therapeutic is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 12 months or more.

In aspects of this embodiment, a therapeutically effective amount of a therapeutic disclosed herein reduces the recovery time in an individual suffering from botulism by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a therapeutic disclosed herein reduces signs/symptoms of botulism in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a therapeutic disclosed herein signs/symptoms of botulism in an individual with the disease by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Method of Treatment of Systemic Botulism (BoNT-A)

In this example, a patient suffers from botulism after returning from overseas travel. The patient complains of weakness, blurred vision, fatigue and difficulty speaking. Other symptoms that develop include weakness of the arms, chest muscles and legs. A health care provider avers that the patient is suffering from botulism (BoNT-A) based on ruling out other ailments and confirmation by fluid samples. The provider suspects that the patient consumed tainted food and suggests BoNT/E to improve the speed of recovery.

The BoNT/E is provided in a solution for intravenous administration. Typical doses of BoNT/E range from 1,000-30,000 total units given in a single treatment. However, a person having ordinary skill in the art of medicine would be able to determine, without undue experimentation, the appropriate dosages and routes of administration of the methods of the disclosure described herein.

The patient is administered a single dose of BoNT/E solution intravenously (10,000 Units). The healthcare provider continues to monitor the extent of the muscle weakness/fatigue as the patient improves. The signs/symptoms of botulism are monitored during the course of treatment. A second dose of BoNT/E solution can be administered if there is no significant reduction in the symptoms. As can be appreciated, the treatment regime can be altered and/or extended based on the patient's symptoms and extent of improvement.

### Example 2

### Method of Treatment of Systemic Botulism (BoNT-A)

In this example, a patient suffers from botulism after consuming tainted food. Because the botulism is advanced, the patient relies on a ventilator. A health care provider confirms systemic botulism using fluid samples. The provider suggests intravenous administration of BoNT/E to improve the speed of recovery.

The BoNT/E is provided in a solution for intravenous administration. As above, typical doses of BoNT/E range from 1,000-30,000 total units given in a single treatment. The patient is administered a single dose of BoNT/E solution intravenously (a total of 10,000 Units).

In a typical scenario, a patient may rely on a ventilator for several months as they recover from systemic botulism. The methods described herein allow administration of large doses of BoNT/E to systemically treat the disease. In this example, the patient improves dramatically within ten days and is removed from the ventilator within two weeks of treatment.

### Example 3

### Method of Treatment of Localized Botulism (BoNT-A)

Botulinum toxin (BOTOX) is often used as a cosmetic treatment. It can be injected to reduce the appearance of skin wrinkles. It has also been approved as a treatment for various health issues, including eyelid spasms, excessive sweating, some bladder disorders, and migraine.

In this example, a patient suffers from botulism after receiving injections of BOTOX for cosmetic reasons. The patient complains of weakness, blurred vision, fatigue and difficulty speaking. Other symptoms that develop include weakness of the arms, chest muscles and legs. A health care provider avers that the patient is suffering from botulism (BoNT-A) because of a series of BOTOX injections received within the past 72 hours.

In this example, BoNT/E is reconstitution from the lyophilized powder using common techniques. Typical doses BoNT/E range from 60-400 total units given in a single treatment by injection. The course of treatment is summarized below. However, a person having ordinary skill in the art of medicine would be able to determine, without undue experimentation, the appropriate dosages and routes of administration of the used in the methods of the disclosure described herein.
Dose per Each Injection=100 Units
Injection Volume per Each Injection=0.3 mL
Number Injections per Each Treatment Session=4 injections
Dose (U) per Each Treatment Session=400 Units (4 injections×100 Units)
Cumulative EFP Dose=1200 Units (3 treatment sessions×400 Units)

In this example, the BoNT/E solution is injected into four different areas where the patient received the cosmetic BOTOX injections. The injections are repeated for a total of three treatment sessions. The signs/symptoms of botulism are monitored during the course of treatment. As can be appreciated, the treatment regime can be altered and/or extended based on the results.

In typical scenarios when Botox injections lead to infection, a patient must wait several weeks for their condition to improve. The methods described herein allow intravenous administration of large doses of BoNT/E to systemically treat the disease. In this example, the patient improves dramatically within five days and is asymptomatic within two weeks of treatment.

### Example 4

### Method of Treatment of Systemic Botulism (BoNT-A)

Botulinum toxins pose a major threat as biological weapons because they are extremely potent and lethal; some of the toxins are relatively easy to produce and transport; and people with botulism require prolonged intensive hospital care. Bioterrorism involving deliberate contamination of food with botulinum toxin would produce similar symptoms, but there would be a connection to a common food source. Initially, there are few clues to help distinguish between an intentional and a naturally occurring foodborne botulism outbreak.

In this example, a hospital emergency department experiences an influx of patients with symptoms of botulism (BoNT-A). The patients complain of similar symptoms including weakness, blurred vision, fatigue and difficulty speaking. The more affected patients complain of weakness of the arms, chest muscles and legs. A health care provider suspects that the patients are suffering from botulism. This is confirmed with laboratory analysis of patient samples (i.e., positive identification of Clostridium botulinum and the toxin).

Based on the influx, the healthcare professional suspects that the patients are victims of a large-scale industrial accident, laboratory accident, food contamination or bioterrorism. The patients are evaluated individually and question regarding what they had recently eaten and where they have been geographically. There is no indication of a naturally occurring foodborne botulism outbreak. The authorities are immediately contacted to help protect members of the public who are susceptible to exposure. The healthcare professional begins to triage and treat the patients.

Patients with more severe symptoms (i.e., muscle weakness) are administered BoNT/E intravenously. As described above, typical doses of BoNT/E range from 1,000-30,000 total units given in a single treatment. The patients are initially administered a single dose of BoNT/E solution intravenously (a total of up to 10,000 Units). Thereafter, the patients are carefully observed for signs of improvement. Particular attention is directed toward those who complain of difficulty breathing.

In aspects, the BoNT/E is administered with an antibiotic. Antibiotics are known in the art and include tetracycline antibiotics and related compounds (e.g., chlortetracycline, oxy-tetracycline, demeclocycline, methacycline, doxycycline, minocycline and roli-tetracycline);

Antibiotic/anti-infective agents also include macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g. cloxacillin, dicloxacillin, nafcillin and oxacillin), extended spectrum penicillins (e.g. aminopenicillins such as ampicillin, amoxicillin, and benzathine benzylpenicillin, and antipseudomonal penicillins such as carbenicillin), cephalosporins (e.g. cefadroxil, cefepime, cefpodoxime, cephalexin, cefazolin, cefoxitin, cefotetan, cefuroxime, cefotaxime, ceftazidime and ceftriaxone) and carbapenems such as imipenem, meropenem and aztreonam; aminoglycoside antibiotics such as streptomycin, gentamicin, tobramycin, amikacin and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole and sulfamethoxazole; silver-based active agents such as elemental silver, silver ions and salts, and silver coordination compounds; quinolone antibiotics such as levofloxacin, trovafloxacin, ciprofloxacin, nalidixic acid and ofloxacin; anti-mycobacterials such as isoniazid, rifampin, rifabutin, ethambutol, pyrazinamide, ethionamide, aminosalicylic and cycloserine; nitrofurans such as nitrofurazone and nitrofurantoin.

The therapeutic methods described herein can include the step of administering drug product (i.e., BoNT/E) at a pharmaceutically effective amount. The total dose should be determined through appropriate medical judgment by a physician and administered once or several times. The specific therapeutically effective dose level for any particular patient may vary depending on various factors well known in the medical art, including the kind and degree of the response to be achieved, concrete compositions according to whether other agents are used therewith or not, the patient's age, body weight, health condition, gender, and diet, the time and route of administration, the secretion rate of the composition, the time period of therapy, other drugs used in combination or coincident with the composition disclosed herein, and like factors well known in the medical arts.

In still another aspect, the present specification provides a use of the pharmaceutical composition in the preparation of drugs for treatment of botulism.

In one embodiment, the dose of the composition may be administered daily, semi-weekly, weekly, bi-weekly, or monthly. The period of treatment may be for a week, two weeks, a month, two months, four months, six months, eight months, a year, or longer. The initial dose may be larger than a sustaining dose. In one embodiment, the dose ranges from a weekly dose of at least 0.01 mg/kg, at least 0.25 mg/kg, at least 0.3 mg/kg, at least 0.5 mg/kg, at least 0.75 mg/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, or at least 30 mg/kg. In one embodiment, a weekly dose may be at most 1.5 mg/kg, at most 2 mg/kg, at most 2.5 mg/kg, at most 3 mg/kg, at most 4 mg/kg, at most 5 mg/kg, at most 6 mg/kg, at most 7 mg/kg, at most 8 mg/kg, at most 9 mg/kg, at most 10 mg/kg, at most 15 mg/kg, at most 20 mg/kg, at most 25 mg/kg, or at most 30 mg/kg. In a particular aspect, the weekly dose may range from 5 mg/kg to 20 mg/kg. In an alternative aspect, the weekly dose may range from 10 mg/kg to 15 mg/kg.

The present specification also provides a pharmaceutical composition for the administration to a subject. The pharmaceutical composition disclosed herein may further include a pharmaceutically acceptable carrier, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical composition including the BoNT/E disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a colorant, and a flavorant. For injectable preparations, the carrier may include a buffering agent, a preserving agent, an analgesic, a solubilizer, an isotonic agent, and a stabilizer. For preparations for topical administration, the carrier may include a base, an excipient, a lubricant, and a preserving agent.

The disclosed compositions may be formulated into a variety of dosage forms in combination with the aforementioned pharmaceutically acceptable carriers. For example, for oral administration, the pharmaceutical composition may be formulated into tablets, troches, capsules, elixirs, suspensions, syrups or wafers. For injectable preparations, the pharmaceutical composition may be formulated into an ampule as a single dosage form or a multidose container. The pharmaceutical composition may also be formulated into solutions, suspensions, tablets, pills, capsules and long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate and mineral oils. In addition, the pharmaceutical formulations may further include fillers, anti-coagulating agents, lubricants, humectants, flavorants, and antiseptics.

Further, the pharmaceutical composition disclosed herein may have any formulation selected from the group consisting of tablets, pills, powders, granules, capsules, suspensions, liquids for internal use, emulsions, syrups, sterile aqueous solutions, non-aqueous solvents, lyophilized formulations and suppositories.

The composition may be formulated into a single dosage form suitable for the patient's body, and preferably is formulated into a preparation useful for peptide drugs according to the typical method in the pharmaceutical field so as to be administered by an oral or parenteral route such as through skin, intravenous, intramuscular, intra-arterial, intramedullary, intramedullary, intraventricular, pulmonary, transdermal, subcutaneous, intraperitoneal, intranasal, intracolonic, topical, sublingual, vaginal, or rectal administration, but is not limited thereto.

The composition may be used by blending with a variety of pharmaceutically acceptable carriers such as physiological saline or organic solvents. In order to increase the stability or absorptivity, carbohydrates such as glucose, sucrose or dextrans, antioxidants such as ascorbic acid or glutathione, chelating agents, low molecular weight proteins or other stabilizers may be used.

The administration dose and frequency of the pharmaceutical composition disclosed herein are determined by the type of active ingredient, together with various factors such as the disease to be treated, administration route, patient's age, sex, gender, body weight, and disease severity.

The total effective dose of the compositions disclosed herein may be administered to a patient in a single dose or may be administered for a long period of time in multiple doses according to a fractionated treatment protocol. In the pharmaceutical composition disclosed herein, the content of active ingredient may vary depending on the disease severity. Preferably, the total daily dose of the peptide disclosed herein may be approximately 0.0001 μg to 500 mg per 1 kg of body weight of a patient. However, the effective dose of the composition is determined considering various factors including patient's age, body weight, health conditions, gender, sex, disease severity, diet, and secretion rate, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical composition may be administered alone or in combination or coincident with other pharmaceutical formulations showing prophylactic or therapeutic efficacy.

Given the teachings and guidance provided herein, those skilled in the art will understand that a formulation described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

In various embodiments, a formulation can include, without limitation, combinations of bioactive agents (such as viruses, proteins, antibodies, peptides and the like as described herein) in the formulation. For example, a formulation as described herein can include a single bioactive agent for treatment of one or more conditions. A formulation as described herein also can include, in an embodiment, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition. The composition can be a solid, a semi-solid or an aerosol and a pharmaceutical compositions is formulated as a tablet, geltab, lozenge, orally dissolved strip, capsule, syrup, oral suspension, emulsion, granule, sprinkle or pellet.

The doses of the BoNT/E can be expressed in mg per injection such as from about 0.001 mg to 0.5 mg per injection, about 0.01 mg to about 5 mg per injection, or about 0.005 mg to about 0.1 mg, or about 0.005 mg, 0.04 mg, or 0.07 mg per injection. The collagenase mixture may be in the form of a pharmaceutical formulation comprising the collagenase and pharmaceutically acceptable excipients.

The doses of the BoNT/E can be expressed in units that are administered. For example, BoNT/E can have a specific activity of about 1,000 units/mg to about 2,500 units/mg, or about 1,500 units/mg, or about 1,750 units/mg, or about 2,000 units/mg, or about 2,500 units/mg, or about 1,000 units/0.58 mg, or 1,724 units/mg wherein “mg” refers to the amount of units present in a composition (as distinct from excipients and other constituents). Accordingly, the present invention contemplates injecting about 100 units to about 500 units per treatment session, or about 1,000 units to about 2,500 units per treatment session.

In another embodiment, the dose of BoNT/E per injection is about 50 units to about 2,500 units, or about 85 units to about 2,000 units, or about 150 units to about 1,750 units, or about 200 units to about 1,500 units, or about 300 units to about 1,250 units, or about 500 units to about 1,000 units.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors. Syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, ocular, dermal, or pulmonary, as appropriate.

In certain embodiments, kits can comprise, without limitation, one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiments the kits can contain a lyophilate and a sterile diluent for reconstituting the lyophilate.

In an embodiment, a formulation as described herein can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, and particularly pharmaceutical virus formulations.

Compositions in accordance with embodiments described herein have desirable properties, such as desirable solubility, viscosity, syringeability and stability. Lyophilates in accordance with embodiments described herein have desirable properties, as well, such as desirable recovery, stability and reconstitution.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9.

In an embodiment, the pH of the pharmaceutical formulation is from about 3 to about 9, about 4 to about 9, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about about 3 to about 8, about 3 to about 7, about 3 to about 6, about 3 to about 5, about 3 to about 4, about 7 to about 8, about 7 to about 9, about 7 to about 10.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A method of treating a subject with botulism, the method comprising administering a therapeutic amount of Type E Botulinum toxin.

2. The method of claim 1, wherein the botulism causes one or more of dysphagia, difficulty breathing, muscle weakness, double vision, drooping of eyelids, loss of facial expression, dry mouth and throat, postural hypotension, constipation, nausea, vomiting and difficulty with talking.

3. The method of claim 1, wherein the botulism is infant botulism, wound botulism or results from foodborne botulism.

4. The method of claim 1, wherein the botulism is the result of exposure to one or more of Type A Botulinum toxin Type C1 Botulinum toxin or Type C2 Botulinum toxin.

5. The method of claim 1, wherein the botulism is the result of therapeutic or cosmetic use of Botulinum toxin.

6. The method of claim 1, further comprising administration of an antitoxin and/or an antibiotic to the subject.

7. The method of claim 1, wherein the administration comprises intravenous injection.

8. The method of claim 1, wherein the administration comprises multiple injections at a plurality of sites.

9. The method of claim 8, wherein each of the multiple injections is comprised of a volume of about 0.2 ml or less.

10. The method of claim 1, wherein about 1,000 to 20,000 Units of Type E Botulinum toxin in solution are administered intravenously to the subject.

11. The method of claim 1, wherein the Type E Botulinum toxin is injected using a syringe with a rack and pinion component.

12. A method of treating botulism in a patient comprising steps of:
a) identifying a patient with signs and/or symptoms of botulism,
b) administering a therapeutic amount of Type E Botulinum toxin to the subject,
c) monitoring signs and/or symptoms of botulism,
d) repeating steps b) and c) until the signs and/or symptoms of botulism subside.

13. The method of claim 12, wherein the signs and/or symptoms include one or more of dysphagia, difficulty breathing, muscle weakness, double vision, drooping of eyelids, loss of facial expression, dry mouth and throat, postural hypotension, constipation, nausea, vomiting and difficulty with talking.

14. The method of claim 12, wherein the botulism is foodborne botulism, infant botulism or wound botulism.

15. The method of claim 12, wherein the botulism is the result of exposure to one or more of Type A Botulinum toxin, Type C1 Botulinum toxin or Type C2 Botulinum toxin.

16. The method of claim 12, wherein the botulism is the result of therapeutic or cosmetic use of botulinum toxin.

17. The method of claim 12, further comprising a step of confirming a presence of Clostridium botulinum and/or botulinum toxin in the subject.

18. The method of claim 12, further comprising a step of administering an antitoxin and/or an antibiotic to the subject.

19. The method of claim 12, wherein the therapeutic amount of Type E Botulinum toxin is administered at multiple sites.

20. The method of claim 16, wherein the therapeutic amount of Type E Botulinum toxin is administered intravenously.
